(19) United States
(12) Patent Application Publication
(10) Pub. No.: US 2026/0056128 A1
(43) Pub. Date: Feb. 26, 2026

(54) RAMAN SPECTROSCOPY SYSTEMS FOR VIRAL DETECTION

(71) Applicant: NOMAD IP I LLC, New York, NY (US)

(72) Inventors: Rupesh Chaturvedi, Fairfax, VA (US); Reem Mahrat, Pleasanton, CA (US); Douglas W. Thomas, Pleasanton, CA (US); Viral Shukla, Pleasanton, CA (US); William D. Howell, New York, NY (US)

(73) Assignee: NOMAD IP I LLC, New York, NY (US)

(21) Appl. No.: 19/105,777

(22) PCT Filed: Aug. 25, 2023
(86) PCT No.: PCT/US2023/072892

§ 371 Date: Feb. 21, 2025

Related U.S. Application Data
(60) Provisional application No. 63/401,028, filed on Aug. 25, 2022.

(51) Int. Cl. G01N 21/65; B01L 3/00; G01N 33/487

(52) U.S. Cl. CPC G01N 21/65; B01L 3/5085; G01N 33/48778; B01L 2200/04; B01L 2300/0848; G01N 2201/0221

(57) ABSTRACT

A cartridge and a system for detecting viruses are described. The cartridge includes a cover, a base, a substrate configured to detect a virus, a sealing element, and a padding element. The cover has a top surface, an inner bottom surface, and a well having a hydrophobic wall extending from the top to the bottom surface. The well has a larger bore size at the top surface than a bore size at the bottom surface. The base is sealingly coupled to the cover to form an inner compartment. The scaling element is disposed within the inner compartment against the inner surface of the cover around the well. The padding element is disposed within the inner compartment on an inner surface of the base to support the substrate so that a sample of bodily fluid can form a pool within the well and stay in contact with the substrate.

### CROSS-REFERENCE TO RELATED APPLICATIONS

This application is a U.S. National Phase application based on International Patent Application No. PCT/US2023/072892, filed on Aug. 25, 2023, which claims the benefit of U.S. Provisional Application No. 63/401,028, filed on Aug. 25, 2022, each of which is incorporated by reference herein in its entirety.

### FIELD OF DISCLOSURE

The present disclosure relates to a portable device for carrying bio-samples or other chemical samples for viral, bacterial, or other testing purposes. For example, the device can store and transport the bio-samples to perform Raman spectroscopy.

### BACKGROUND OF THE DISCLOSURE

Pandemics (e.g., COVID-19) have demonstrated that preventing new variants from spreading between countries, continents, or between the inside of an establishment (business, operation center, school, etc.) is very challenging. The spread can be unimpeded even when active vaccine deployment, temperature checking, questionnaires, and rapid diagnostics have been utilized. Diagnostics are valuable tools to confirm not only cases where the symptoms are consistent with infection but also, due to the high rate of infectious non-symptomatic cases and the relatively long prodromal phase for the viruses, find hiding infections before they are spread to other individuals.

Diagnostics and testing for viruses can require training, can be expensive, and time-consuming. In addition, obtaining accurate test results is not guaranteed. For example, the accuracy of PCR is considered the gold standard for sensitivity and its ability to detect viral RNA very early in infection. The speed of the test continues to increase, but it requires a trained professional and expensive equipment to get an accurate result. Its cost is relatively high.

Traveling from one country to another requires fast and accurate results to prevent spreading infections. If travel has been delayed or the individual requires an airport-based test, the cost can be in the few hundred-dollar range. The problem with this approach is that the traveler may have been exposed after the test was given, thus, a negative test may not be accurate for a passenger's true status of infection. Hence, a need remains for means of detecting infections that are cost-effective, faster, and more accurate means that do not require operators with high skills or extensive training.

### BRIEF SUMMARY OF THE DISCLOSURE

One aspect of the present disclosure relates to a device for testing samples such as biological sample, chemical or other samples to determine infections. The device can be a cartridge with one or more wells to receive the biosample. The cartridge can include a cover, a base, a sealing element, and a padding element. The cover has a top surface from which a first side wall, a second side wall, a third side wall, and a fourth side wall extend, an inner bottom surface opposite to the top surface, and a well comprising a hydrophobic wall extending from the top surface to the inner bottom surface. The well has a larger bore size at the top surface than a bore size at the inner surface. The base has an inner surface and sealingly coupled to the cover. The inner surfaces of the cover and the base are surrounded by the first, second, third, and fourth side walls forming an inner compartment. The sealing element is disposed within the inner compartment against the inner surface of the cover around the well. The padding element is disposed within the inner compartment on the inner surface of the base to support and press a substrate configured to detect a virus against the scaling element so that a sample of bodily fluid can form a pool within the well and stay in contact with the substrate.

In many embodiments, the hydrophobic wall of the well are conical in shape with a first top diameter being larger than a second bottom diameter. The first top diameter is in a range from 0.5 mm to 7 mm and the second bottom diameter is in a range from 0.5 mm to 5 mm. The difference between the first top diameter the second bottom diameter is in a range from 0.5 to 3 mm. The depth of the well is in a range from 0.5 mm to 7 mm. The hydrophobic wall is made of or covered with hydrophobic material, the hydrophobic material compatible with the sample of bodily fluid comprising at least one of: Teflon, PET (Polyethylene terephthalate), alkanes or PDMS (Polydimethylsiloxane). The hydrophobic wall is inclined at an angle from 45° to less than 90° with respect to the top surface of the cover to cause the pool of the sample of bodily fluid to form a contact angle between 90° to 150° with respect to the substrate. The sample of the bodily fluid has a surface tension that depends on type of biological fluids and material of cartridge. The hydrophobic wall of the well has a height in a range from 1 mm to 50 mm. The hydrophobic wall of the well and the scaling element define a volume in a range from 1 μL to 20 μL to hold the pool of the sample of bodily fluid in contact with and above the substrate.

In many embodiments, the inner surface of the cover includes a groove to receive the scaling element. The sealing element is at least one of: an O-ring coated with hydrophobic material, or a hydrophobic sealant receivable in the groove. The O-ring extends from the inner surface of the cover into the inner compartment to increase a well volume above the substrate.

In many embodiments, the cover includes a plurality of wells spaced apart from each other. Each well has a hydrophobic wall configured to receive the sample of bodily fluid and have a larger bore size at the top surface than a bore size at the inner surface. The well has a depth that is within a focus range of a spectrometer or an optical device used to detect virus.

In another aspect, a method of testing for a virus is described. The method includes receiving a sample of bodily fluid within a well of a cartridge. The cartridge housing a substrate configured to detect a virus, the well comprising an open end to receive the sample and a hydrophobic wall to allow the sample to travel to the substrate. Further, the method involves directing a light of a particular wavelength that is suitable for Raman spectroscopy from the open end of the well through the sample of the bodily fluid to interact with the substrate. The method further involves receiving reflected light from the substrate after passing through the sample, and analyzing the reflected light to detect a type of the virus. The sample is a bodily fluid. The virus titer in the sample is within the range of 109 to 100 copies/ml saliva.

In some embodiments, directing light through the sample involves placing the cartridge in a Raman Spectrometer. In some embodiments, directing light through the sample involves aligning a handheld Raman Spectrometer over the well. In some embodiments, directing light through the sample involves scanning across the well of the cartridge with x and y movement of the cartridge using a moving cartridge holder platform.

In some embodiments, receiving reflected light involves collecting, via a detector, the reflected light to measure Raman displacement in a range from 800 cm to 3200 cm. In some embodiments, analyzing the reflected light involves capturing displacements in Raman spectrum of the reflected light caused due to molecular interaction between the virus and the substrate. In some embodiments, analyzing the reflected light involves measuring intensity of each Raman shift or displacement within the Raman spectrum.

In some embodiments, the method further involves receiving multiple samples of the bodily fluid, wherein the cartridge comprises a plurality of wells, each well being spaced and isolated from each other, wherein each sample of the multiple samples is received within a well of the plurality of wells in the cartridge, directing the light over each of the wells through the sample within the well; and analyzing the reflected light received from each of the plurality of well to detect different types of viruses.

In another aspect, a system for detecting a virus is described. The system includes a cartridge and a Raman spectrometer. The cartridge includes a base, a cover, and a scaling element. The cover is sealing coupled to the base scaling coupled to the cover forming an inner compartment therebetween. The cover includes a well having a hydrophobic wall and a larger bore size at a top side than a bore size at a bottom side to hold a sample of bodily fluid within the well in contact with and above a substrate used to detect a virus. The scaling element is disposed within the inner compartment around the well and above the substrate. The Raman spectrometer is configured to: direct light of a particular wavelength toward the well; receive reflected light from the substrate after passing through the sample of bodily fluid; and analyze a spectrum of the reflected light to determine a type of the virus. In some embodiments, the hydrophobic wall of the well comprises a first top diameter in a range from 0.5 mm to 7 mm and a second bottom diameter in a range from 0.5 mm to 5 mm to facilitate generation of Raman spectrum by a Raman spectrometer.

In some embodiments, devices and methods herein including a cartridge with a sensor, a machine to read the signal from the sensor, and the use of that signal to determine and inform a subject of their status for a viral infection. The device and methods herein provide various advantages. For example, the cartridge housing a virus or other infection detection substrate and method for detecting the same can enable rapidly detecting (e.g., in less than 5 minutes or less than 1 minute), delineating (classify/identify), and quantifying reparatory viral infection from a sample (e.g., a saliva sample) using a Raman spectroscopic method. The cartridge facilitates case of handling of the saliva samples and enables the use of a handheld Raman spectrometer to detect viruses, if any, in the saliva samples. Thus, personnel with little to no training can use the cartridge for virus detection e.g., at high-traffic public places such as airports, bus stations, etc. Accordingly, a system employing the cartridge herein can generate real-time data and analysis to provide a gateway for yes or no passage based on a positive or negative result for an infectious disease. Furthermore, a spectral scan can be read from the saliva sample in the cartridge. The cartridge can be configured to include surface-enhanced Raman spectroscopy (SERS) substrates, which amplifies the Raman spectrophotometric light scattering result from a laser (e.g., 785 nm), or other commercially available substrates for detecting infections (e.g., virus, bacteria, etc.). This spectral information can be communicated to a cloud-based machine learning application that searches for one or more matches in the cloud-based description library. The returned quantitative matches can be communicated to a user or the results can be stored in a database for later population research.

The methods and compositions disclosed herein can be used to determine accurately the infection, either symptomatic or asymptomatic. This is particularly useful to be deployed at places where rapid, and real-time diagnostics is required, for example, at an airport gateway. For example, this technology can rapidly determine the viral infection status of a passenger while the he or she is in line at the security gate.

### BRIEF DESCRIPTION OF THE DRAWINGS

The accompanying drawings, which are incorporated in and constitute a part of the specification, illustrate one or more embodiments and, together with the description, explain these embodiments. The accompanying drawings have not necessarily been drawn to scale. Any values dimensions illustrated in the accompanying figures are for illustration purposes only and can or cannot represent actual or preferred values or dimensions. Where applicable, some or all features cannot be illustrated to assist in the description of underlying features. In the drawings:

FIG. 1A illustrates an example cartridge in accordance with some embodiments of the present disclosure.

FIG. 1B illustrates another example cartridge in accordance with some embodiments of the present disclosure.

FIG. 2 is an exploded view of the cartridge of FIG. 1A.

FIG. 3 illustrates an underside of a cover of the cartridge of FIG. 2.

FIG. 4 is a cross-section view of the cartridge showing a saliva pool in a well of the cartridge of FIG. 2.

FIG. 5 is a top view picture of the cartridge showing a saliva pool in a well of the cartridge of FIG. 2.

FIG. 6 illustrates a saliva pool spread across a substrate.

FIG. 7 is an exploded view of another example cartridge having multiple wells in accordance with some embodiments.

FIG. 8 illustrates (A) a top view and (B) a side view of the cartridge of FIG. 7.

FIG. 9 is a schematic of Raman spectrometry performed on the saliva pool in the cartridge of FIG. 1A, 1B, or 7.

FIG. 10 is a flow chart of a method for testing a virus using a cartridge, in accordance with some embodiments.

### DETAILED DESCRIPTION OF THE DISCLOSURE

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

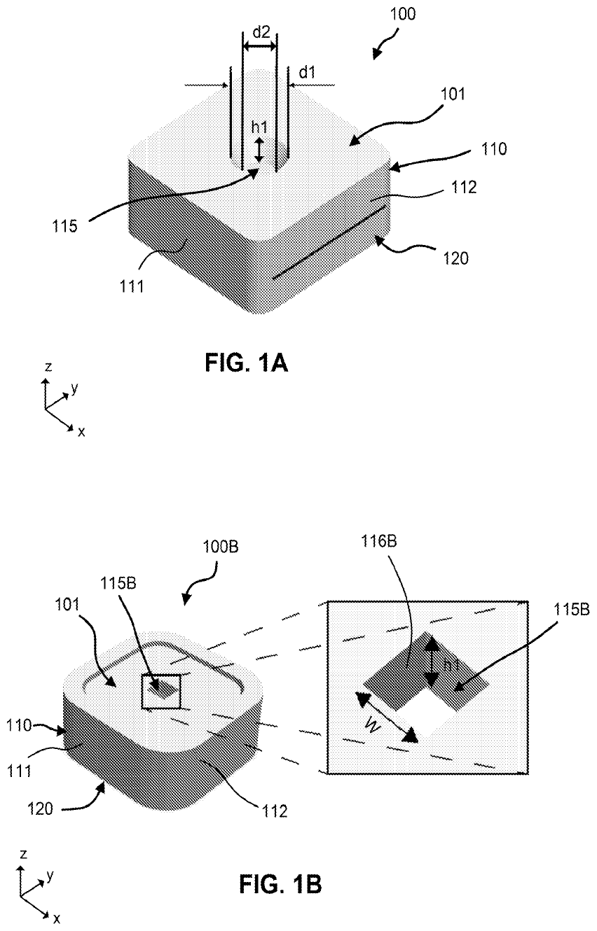

Referring now to the drawings wherein like reference numerals are used to identify identical components in the various views, FIGS. 1A, 2, and 3 illustrate different views of an example cartridge in accordance with many embodiments of the present disclosure. A cartridge 100 includes a cover 110, a base 120, a sealing element 130, a substrate 140, and a padding element 150. The sealing element 130, the substrate 140, and the padding element 150 can be stacked on top of each other such that the padding element 150 is next to the base 120 and the sealing element 130 is next to the cover 110. The substrate 140 is disposed between the sealing element 130 and the padding element 150. The substrate 140 can be any substrate configured to test/detect presence or absence of a virus (e.g., respiratory viruses such as the influenza viruses, respiratory syncytial virus, parainfluenza viruses, metapneumovirus, rhinovirus, coronaviruses, adenoviruses, and bocaviruses) in a biological sample. For example, the substrate can be a surface-enhanced Raman spectrophotometry (SERS) chip (e.g., silver nano-structure sensor), such as disclosed in “Label-Free Spectroscopic SARS-COV-2 Detection on Versatile Nanoimprinted Substrates” by Paria et al., Nano Lett. 2022 May 11; 22 (9): 3620-3627, which is incorporated herein by reference in its entirety. Additional suitable substrates include gold and silver based nano structures. In some embodiments, the substrate 140 does not comprise aluminum. Such substrates can be fragile, as such components in the cartridge 100 provide rigidity to the substrate 140 as well as improving case of handling of the substrate. The cartridge 100 can be printed with a 3D printer, mold manufactured using plastic or and any other suitable material, or other manufacturing techniques may be employed.

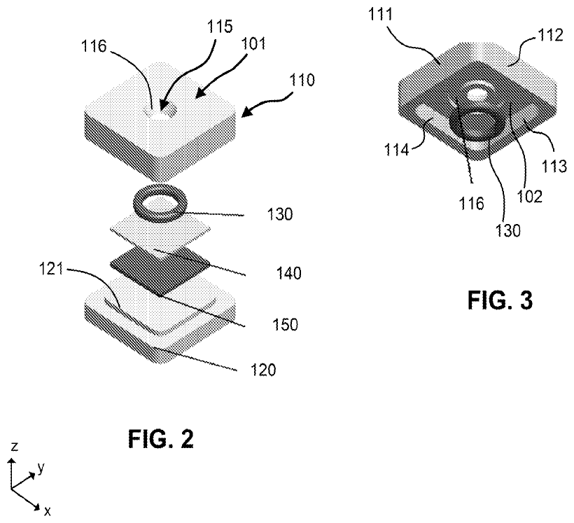

In many embodiments, a cover 110 can include a top surface 101, an inner bottom surface 102 opposite to the top surface 101 (see FIG. 2), and a well 115 extending from the top surface 101 to the inner bottom surface 102. The cover 110 can have any geometrical shape compatible with the base 120 that can make the cartridge 100 portable. For example, the cover 110 can have an open box-like shape such that a first side wall 111, a second side wall 112, a third side wall 113, and a fourth side wall 114, which extend from the top surface 101. The bottom end can be open and coupled to the base 120 to form an enclosed inner compartment within which one or more components such as the substrate 140, the padding element 150, and the sealing element 130 can be disposed so that a portion of the substrate 140 is accessible only via the well 115.

The well 115 can include a hydrophobic wall 116 extending from the top surface 101 to the inner bottom surface 102. The well 115 can have a larger bore size at the top surface 101 than a bore size at the inner surface 102. In some embodiments, the hydrophobic wall 116 of the well 115 can be conical in shape with a first top diameter d1 being larger than a second bottom diameter d2. As an example, the first top diameter d1 is in a range from 0.5 mm to 7 mm, 1 mm to 5 mm, 1 mm to 2 mm, or other ranges. The second bottom diameter d2 is in a range from 0.5 mm to 5 mm, 1 mm to 4 mm, 1 mm to 2 mm or other ranges. In some embodiments, a difference between the first top diameter d1 the second bottom diameter d2 is in a range from 0.5 to 3 mm, 1 mm to 2 mm or other ranges. The well 115 can have a depth h1 defined according to a volume of sample to be deposited in the well 115. For example, the depth h1 can be in a range from 0.5 mm to 7 mm based on volume of samples ranging from 5 ul to 25 ul. In some embodiments, the depth h1 of the well 115 can be a function a parameter of an optical device (e.g., spectrometer) used to detect infection in the sample. For example, the depth h1 of the well 115 can be such that it remains within a focus range of the optical device.

The hydrophobic wall 116 can be inclined at an angle from 45° to less than 90° with respect to the top surface 101 of the cover 110 to cause the pool of the sample of bodily fluid to form a contact angle between 90° to 150° with respect to the substrate 140. As other example, the hydrophobic wall 116 can be inclined at an angle between 50° to 80°, 50° to 60°, or other ranges, and the contact angle can be between 100° to 140°, 120° to 145° or other ranges. The sample of the bodily fluid has a surface tension that depends on type of biological fluids and material of cartridge 100. Such hydrophobic wall 116 allows the bodily fluid to slide and accumulate over the substrate 140. If the well 115 does not have such hydrophobic wall, the surface tension of the fluid (e.g., saliva) exerted on the wall can prevent the fluid from reaching the substrate 140, resulting in unsuccessful virus detection.

In many embodiments, the hydrophobic wall 116 can be made of or covered with hydrophobic material. The hydrophobic material is compatible with a sample (e.g., bio sample such as saliva, chemical sample, etc.) to be deposited in the well 115. For example, the hydrophobic wall 116 material may be at least one of Teflon, PET (Polyethylene terephthalate), alkanes, PDMS (Polydimethylsiloxane) or other materials, or any combinations thereof.

The base 120 can have an inner surface 121 configured to support the substrate 140 and/or the padding element 150. Edges of the base 120 can be sealingly coupled to the cover 110. As an example, the base 120 can be made of plastic and can include open box-like structure having walls configured to sealing couple with the side walls 111-114 of the cover 110. The inner surfaces of the cover 110 and the base 120 surrounded by the first, second, third, and fourth side walls 111-114 to form an inner compartment. Within the inner compartment, the sealing element 130 can be disposed against the inner surface 102 of the cover 110 around the well 115 to prevent leakage of the fluid sample (e.g., biological or chemical sample) from the well 115. In some embodiments, the sealing element 130 can also include hydrophobic surface in contact with the fluid sample. As an example, the sealing element 130 can be a gasket rubber ring or any hydrophobic sealant. The sealing element 130 and the hydrophobic wall 116 of the well 115 can define a volume in a range from 1 μL to 20 μL to hold the pool of the sample of bodily fluid in contact with and above the substrate 140. In some examples, the height of the well 115 from the top surface 101 to the top of the substrate 140 can be from 0.5 mm to 50 mm.

In an illustrated embodiment, see FIG. 3, the inner surface 102 of the cover 110 includes a groove 116 to receive the sealing element 130. The sealing element 130 can be at least one of an O-ring coated with hydrophobic material, or a hydrophobic sealant receivable in the groove 116. The O-ring extends from the inner surface 102 of the cover 110 into the inner compartment. The sealing element 130 can be configured to increase a well volume above the substrate 140, for example, by increasing thickness of the sealing element 130, the distance between the inner surface 112 of the cover 110 and the substrate 140 can be increased.

The padding element 150 can disposed within the inner compartment on the inner surface 121 of the base 120 to support the substrate 140. The padding element 150 can be made of soft material such as rubber, fabric, cotton, or other padding material. The padding element 150 can absorb any excess liquid overflowing or seeping from the well. The padding element 150 can have a thickness such that it can lightly press the substrate 140 against the sealing element 130 so that a sample of bodily fluid can form a pool within the well 115 and stay in contact with the substrate 140. Thus, the padding element 150 can facilitate filling any gaps within the inner compartment of the cartridge 100 while snugly fitting the substrate 140 and the sealing element 130 to receive fluid sample from the well 115 so that there is no leakage of the fluid sample.

It can be understood the present disclosure is not limited to a circular shaped well 115 and other geometric well shapes are possible. For example, well 115 can be V-shaped or U-shaped. As another example, FIG. 1B illustrates another example cartridge 100B having a well 115B with a rectangular or square shape having a width W and a depth h1. The shape of the well can be designed based on a delivery mechanism used to deposit sample within the well, manufacturing case, or other factors.

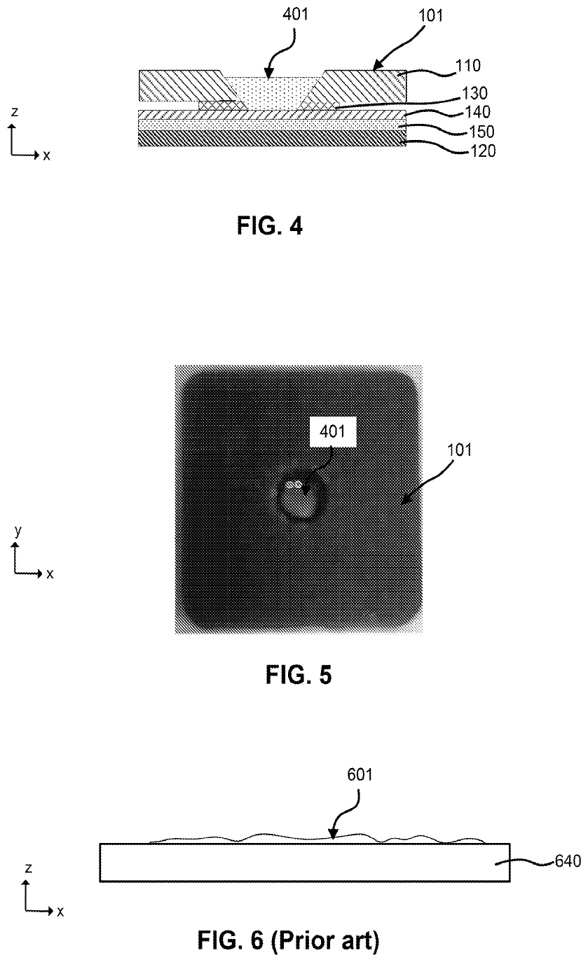

FIG. 4 is cross-section view of the cartridge 100. FIG. 5 is a top view of a manufactured cartridge 100 showing a saliva pool, as an example of the sample deposited in the well 115 of the cartridge 100. As shown in FIG. 4 and FIG. 5, the saliva pool 401 is formed on the substrate 140 and has sufficient height above the substrate 140 due to the well 115 that advantageously allows safely storing or transporting the saliva pool 401 in the cartridge 100 without spilling the saliva pool 401.

The cartridge herein provides several advantages. For example, a sample, e.g., the saliva pool 401 can be collected from a patient at a first location e.g., in an office and transported to a testing location e.g., a laboratory for testing or detecting virus. The cartridge 100 also facilitates testing using a handheld medical testing devices such as a handheld optical device that can project light on the top of the saliva pool 401 and receive reflected light from the substrate 140. Signals in the reflected light can be further analyzed to enable detecting a virus. As compared to conventional sensor scanning area that is large as the saliva sample typically thinly spreads across the substrate, the well 115 of the cartridge 100 disclosed herein advantageously reduces the scanning area that the sample occupies and thus substantially increase the speed of the testing. The cartridge helps in depositing the saliva sample from saliva collection applicator. The hydrophobic walls of the well 115 provide a sliding motion to the saliva sample that all the sample get to sensor in an even manner. Thus cartridge 100 enables several medical or chemical applications at an airport, bus station, train stations, offices, etc. where instant testing and results may be desired to avoid contamination of surrounding or infection from spreading to other people. Furthermore, in some embodiments, the sample can be collected and advantageously stored (e.g., in dark, sterile environment, cold storage or other storing environment) from 24-72 hours before making any measured or virus detection. The substrate 140 can be a silicon wafers, which are very thin and easy get deform. Thus cartridge 100 provides rigidity and support to the substrate 140. The well depth can be utilized to set a minimum and maximum distance from the substrate 140 to an optical probe of Raman spectrometer. The cartridges herein can facilitate the measurements in liquid. This is advantageous as compared to the conventional method of detection which requires the biological materials to be dried when deposited on the substrate before measurements.

On the contrary, existing testing approaches involve directly depositing the saliva on a substrate 640, as shown in FIG. 6. In this case, the saliva pool spreads unevenly across the substrate 640. As such, an amount of sample that can deposited on the substrate 640 is small e.g., less than 2 μl as adding more fluid sample will simply spread and roll off the substrate 640. Furthermore, the saliva pool can fall off the substrate 640 while transporting or during testing. In several case, as the saliva spreads over the substrate 640, sufficient thickness of saliva pool may not be available thereby affecting test results e.g., generating frequent false negatives while people may be infected. Thus, even if same substrate 640 may be used for detecting viruses, the cartridge 100 can not only provide more reliable results due to sufficient saliva pool thickness but also improves portability.

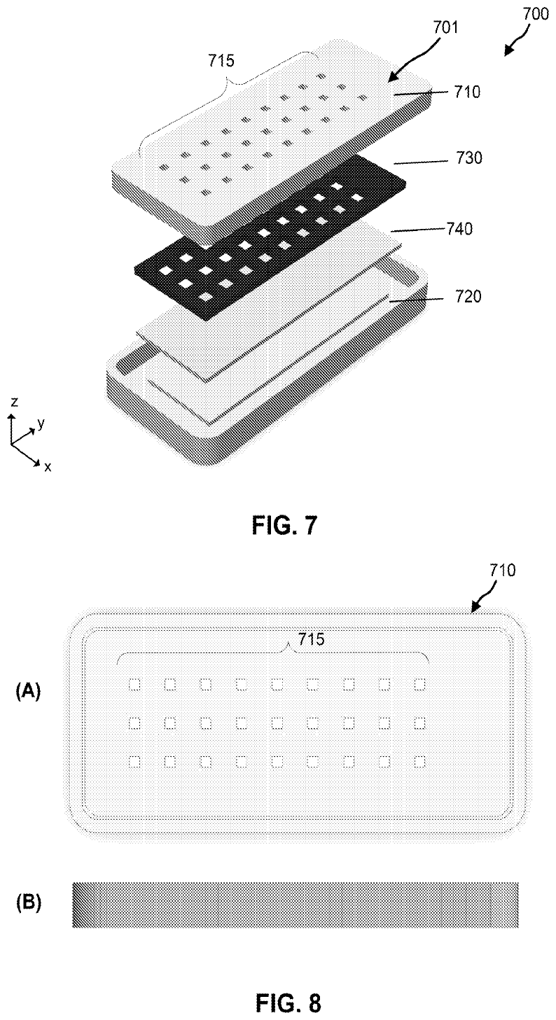

FIG. 7 through FIG. 8 illustrates another embodiment of a cartridge 700. FIG. 7 is an exploded view of the cartridge 700 having multiple wells. FIG. 8 illustrates (A) top view and (B) a side view of the cartridge 700. The cartridge 700 can be a rectangular box-type shape including a cover 710 and a base 720 forming an inner compartment. A sealing element 730 and a substrate 740 can be configured to be disposed within the inner compartment. The cover 710 can include a plurality of wells 715 spaced apart from each other. Similar to wells 115, each of the wells 715 can include a hydrophobic wall configured to receive the sample of bodily fluid and have a larger bore size at a top surface 701 of the cover 710 than a bore size at an inner surface. In the illustrated embodiment, the plurality of wells 715 are distributed in an array (e.g., 2×2, 2×3, 3×10, etc.). The sealing element 730 can have a shape corresponding to the top surface 701. For example, the sealing element 730 can be rectangular in shape with a plurality of holes corresponding to the wells 715. The scaling elements 730 and the holes therein are configured to isolate the wells 715 from each other so that multiple samples may be deposited within the wells 715 so facilitate simultaneous testing of multiple samples using the same cartridge 700 thus improving the speed and accuracy of testing results.

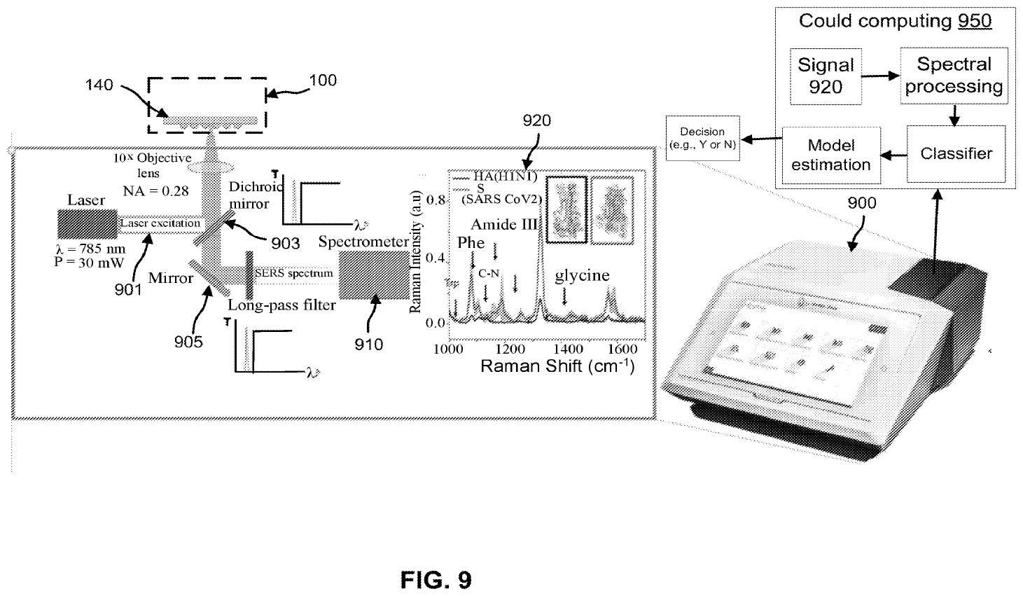

FIG. 9 is a schematic of Raman spectrometry performed on the saliva pool in the cartridge of FIG. 1A, 1B, or 7. A Raman spectrometer 900 can be configured to direct light 901 of a particular wavelength toward the well 115 (see FIG. 1A) of the cartridge 100. For example, a beam splitter including a mirror 903 can be used to direct the light 901 towards the well 115 of the cartridge 100. The reflected light from the substrate 140 can be received by the spectrometer 910 after passing through a sample of bodily fluid. In an example, the reflected light can be directed via another mirror 905 and passed through a low pass filter towards the spectrometer 920. Further, a spectrum 920 of the reflected light can be analyzed to determine a type of the virus. For example, signals in the spectrum 920 may be compared with signatures of one or more viruses to detect a particular virus. The spectrometer 900 can include a detector with ability to collect scattered light to measure Raman sift or displacement with range from 800 to 3200 cm. The spectrometer 900 can be configured to measure the Raman intensity of each Raman shift. Further, the spectrometer 900 can be configured to send the Raman intensity and Raman shift data to a cloud storage or cloud-based software for analysis.

Applying Raman spectroscopic method and the cartridge 100 can be used to rapidly detect (e.g., in less than 1 minute), delineate (classify/identify), and quantify reparatory viral infection from a saliva sample. In Raman spectroscopy, a spectral scan can be read from the cartridge 100 containing a saliva sample. The cartridge 100 can include an embedded SERS (surface-enhanced Raman spectrophotometry) chip as an example substrate 140, which amplifies the Raman spectrophotometric light scattering result from a laser (e.g., having wavelength 785 nm). The spectral information e.g., signal 920 can be communicated (e.g., using Wi-Fi or other wireless, or wired communication links) to a cloud computing 950 configured to include perform spectral processing, a signal classifier, and a model (e.g., machine learning application) that can search for one or more matches in the cloud-based description library and return quantitative matches. Furthermore, the retuned quantitative matches can be communicated both a spectral reader to a user as well as to a cloud-based SQL searchable database for later population research. For example, a decision made by the model estimation module (e.g., implementing AI) such as virus detected or not, positive or negative test result, or a non-deterministic decision.

Although FIG. 9 illustrates a table mounted device (e.g., a Raman spectrometer), it does not limit the scope of the present disclosure. The cartridge herein facilitates use of handheld device (e.g., a handheld optical device configured to perform Raman spectrometry) for testing of biological samples. Such handheld devices may be used at public places such as airport, bus station, or other public places. Since the sample is held in the well of the cartridge, the sample can be handled by minimally trained personnel in a public place thus enabling mass testing.

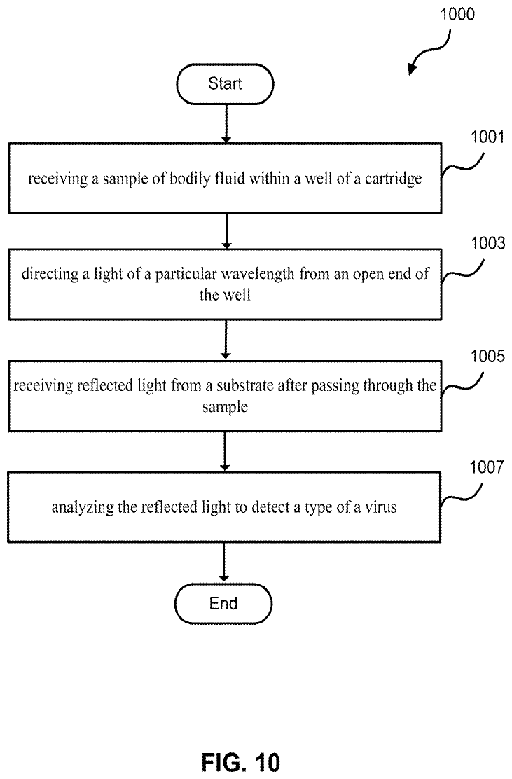

FIG. 10 is a flow chart of a method for testing a virus using the cartridge discussed herein (e.g., the cartridge 100 in FIG. 1A, 100B in FIG. 1B, or 700 in FIG. 7). As an example, a method 1000 for detecting a virus can involve steps 1001, 1003, 1005, and 1007 discussed in detail below.

Step 1001 involves receiving a sample of bodily fluid within a well 115 of a cartridge 100 (see FIGS. 1 and 2). For example, the cartridge 100 houses the substrate 140 configured to detect a virus. The sample of bodily fluid can be received in the well 115 from an open end (e.g., the top surface 101) and a hydrophobic wall 116 allows the sample to travel to the substrate 140 (e.g., see FIG. 4). In some embodiments, the sample is a bodily fluid to be tested for virus or other disease, or chemical process. The titer of the virus in each sample may vary. As an example, the virus titer in the sample can be within the dynamic range of 10⁹ to 100 copies/ml saliva, for example, 10⁸ to 10³ copies/ml, 10⁷ to 10⁴ copies/ml 10⁶ to 10³ copies/ml 10⁵ to 100 copies/ml.

Step 1003 involves directing a light of a particular wavelength from the open end (e.g., at the top surface 101) of the well 115 through the sample of the bodily fluid to interact with the substrate 140. For example, as discussed with respect to FIG. 9, directing light through the sample involves placing the cartridge 100 in a Raman Spectrometer. As another example, directing light through the sample involves aligning a handheld Raman Spectrometer (not illustrated) over the well 115 and directing the light into the well 115 so that the light can travel to the substrate 140 and reflect back to the handheld device. In some embodiments, directing light through the sample involves scanning across the well 115 of the cartridge 100 with x and y movement of the cartridge 100 using a moving cartridge holder platform.

Step 1005 involves receiving reflected light from the substrate 140 after passing through the sample. Receiving reflected light involves collecting, via a detector, the reflected light to measure Raman displacement in a range from 800 cm to 3200 cm. For example, as shown in FIG. 9, the reflected light is received by the spectrometer 920.

Step 1007 involves analyzing the reflected light to detect a type of the virus. For example, analyzing the reflected light involves capturing displacements in Raman spectrum of the reflected light caused due to molecular interaction between the virus and the substrate 140. As an example, analyzing the reflected light can involve measuring intensity of each Raman shift or displacement within the Raman spectrum.

In some embodiments, step 1001 involves receiving multiple samples of the bodily fluid in a cartridge with multiple wells (e.g., the cartridge 700 with wells 715 in FIG. 7), where each well is spaced and isolated from each other. Each sample of the multiple samples can be received within a well of the plurality of wells in the cartridge (e.g., 700). Accordingly, step 1003 may involve directing the light over each of the wells through the sample within the well. The reflected light received from each of the plurality of well can be further analyzed (e.g., by a spectrometer) to detect different types of viruses.

In many embodiments, a machine learning-based analysis software (e.g., a cloud-based software) can compare molecular fingerprints of the spectrograph against a library of spectrographic results to find matches in a virus description in a database. In many embodiments, a device can house a Raman spectral database for the description of respiratory virus in a format that renders them searchable and quantifiable. The database also allows updated viruses descriptions to be uploaded to allow for new searches of archived spectral data. In many embodiments, a system can receive matched/unmatched spectral results and communicate these results back to a display or other interfacing devices. In many embodiments, a system can save spectral and extra-spectral attributes of a searchable database. The database can include saved and linked elements such as viral type found, viral titer, location of finding, destination of individual, date/time of travel, personal attributes (e.g., sex, age), and raw spectral data (e.g., allowing a re-search of the spectral data when new descriptions are uploaded). In many embodiments, a system can generate real-time data and analysis to provide a gateway for yes or no passage based on a positive or negative result for an infectious disease.

In some embodiments, the system may further comprise a Raman spectrometer (for example, 910 in FIG. 9). In some cases, the Raman spectrometer is a Raman microscope, for example, one available from Horiba Instruments Inc., Edison, NJ, USA. In some embodiments, the Raman spectrometer is a handheld Raman spectrometer, for example, one available from Bruker (Camarillo, CA). In some embodiments, the Raman spectrometer can be a custom-made Raman set-up.

The system may also comprise an excitation source (for example, 901 in FIG. 9) includes but is not limited to, illumination source such as a diode laser and an optical fiber laser, dye laser, solid state laser, which provides a light directed to the sample-loaded substrate in the cartridge. The light typically has a wavelength (excitation wavelength) that is suitable for Raman spectrometry. In some embodiments the light has a wavelength of 532, 660, 690, or 785 nm.

In some embodiments, the system may also comprise a data collection and analysis system for collecting the Raman signal produced by the excitation of the substrate and a system for producing the Raman spectra.

The method 1001 can be performed at areas with high foot traffic such as an airport, a bus stations, security gateways, etc. For example, at an airport, a saliva sample may be received in a well of a cartridge (e.g., in the well 115 of the cartridge 100 in FIG. 1) from an airline passenger before passing through the security gate or before boarding a plane. In some examples, saliva samples from multiple passengers may be received within a cartridge with multiple wells (e.g., the wells 715 of the cartridge 700 in FIG. 7). After receiving the sample, testing for infections such as virus can be performed instantly using a testing device (e.g., a handheld spectrometer or a table mounted Raman spectrometer as shown in FIG. 9). For example, a security guard, a nurse, or other medical professional may direct a light from the spectrometer into the well (e.g., 115) of the cartridge (e.g., 100) to reach the substrate (e.g., 140) within the cartridge (100). The light is then reflected from the substrate (e.g., 140) after passing through the saliva sample and received by the spectrometer. Further, an analysis of signals within the reflective light may be performed to detect a type of infection e.g., a virus in the saliva sample. For example, as shown in FIG. 9, the spectrometer (e.g., 920) can receive a reflected light spectrum comprising one or more signatures of a virus characterized by e.g., intensity and frequency data of signal in the spectrum. As discussed herein, the signal may be compared with a database of virus signatures to detect one or more infections. If an infection is detected, the passenger may be informed accordingly. In case of highly contagious injection (e.g., COVID virus) the passenger may be advised to instantly quarantine and prevent boarding the plane thereby preventing spread of the infection and causing a pandemic. In this way, the cartridge (e.g., 100) having the substrate (e.g., 140) configured to detect a particular virus or bacteria (e.g., COVID virus, influenza virus, Zika virus, Mycobacterium tuberculosis bacteria) can quickly (less than two minutes, or less than one minute) and reliably detect infections. Also, advantageously, the cartridge (e.g., 100) facilitates virus detection by a low skilled employee with no to low medical training, e.g., by an airline employee or security guard. Thus, the cartridge herein provides a cost-effective and reliable way to detect infections and prevent mass spread of such infections.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the disclosure and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

What is claimed is:

1. A cartridge, comprising:
a cover having a top surface from which a first side wall, a second side wall, a third side wall, and a fourth side wall extend, an inner bottom surface opposite to the top surface, and a well comprising a hydrophobic wall extending from the top surface to the inner bottom surface, the well having a larger bore size at the top surface than a bore size at the inner surface;
a base having an inner surface and sealingly coupled to the cover,
wherein the inner surfaces of the cover and the base surrounded by the first, second, third, and fourth side walls forming an inner compartment;
a sealing element disposed within the inner compartment against the inner surface of the cover around the well; and
a padding element disposed within the inner compartment on the inner surface of the base to support and press a substrate configured to detect a virus against the sealing element so that a sample of bodily fluid can form a pool within the well and stay in contact with the substrate.

2. The cartridge of claim 1, wherein the hydrophobic wall of the well are conical in shape with a first top diameter being larger than a second bottom diameter.

3. The cartridge of claim 2, wherein the first top diameter is in a range from 0.5 mm to 7 mm and the second bottom diameter is in a range from 0.5 mm to 5 mm.

4. The method of claim 3, wherein the difference between the first top diameter the second bottom diameter is in a range from 0.5 to 3 mm.

5. The method of claim 2, wherein the depth of the well is in a range from 0.5 mm to 7 mm.

6. The cartridge of claim 1, wherein the hydrophobic wall is made of or covered with hydrophobic material, the hydrophobic material compatible with the sample of bodily fluid comprising at least one of: Teflon, PET (Polyethylene terephthalate), alkanes or PDMS (Polydimethylsiloxane).

7. The cartridge of claim 1, wherein hydrophobic wall is inclined at an angle from 45° to less than 90° with respect to the top surface of the cover to cause the pool of the sample of bodily fluid to form a contact angle between 90° to 150° with respect to the substrate, wherein the sample of the bodily fluid has a surface tension that depends on type of biological fluids and material of cartridge.

8. The cartridge of claim 1, wherein the hydrophobic wall of the well has a height in a range from 1 mm to 50 mm.

9. The cartridge of claim 1, wherein the hydrophobic wall of the well and the sealing element define a volume in a range from 1 μL to 20 μL to hold the pool of the sample of bodily fluid in contact with and above the substrate.

10. The cartridge of claim 1, wherein the inner surface of the cover comprises a groove to receive the sealing element.

11. The cartridge of claim 1, wherein the sealing element is at least one of: an O-ring coated with hydrophobic material, or a hydrophobic sealant receivable in the groove.

12. The cartridge of claim 11, wherein the O-ring extends from the inner surface of the cover into the inner compartment to increase a well volume above the substrate.

13. The cartridge of claim 1, wherein the cover comprises a plurality of wells spaced apart from each other, each well having a hydrophobic wall configured to receive the sample of bodily fluid and have a larger bore size at the top surface than a bore size at the inner surface.

14. The cartridge of claim 1, wherein the well has a depth that is within a focus range of a spectrometer or an optical device used to detect virus.

15. A method of testing for a virus, the method comprising:
receiving a sample of bodily fluid within a well of a cartridge, the cartridge housing a substrate configured to detect a virus, the well comprising an open end to receive the sample and a hydrophobic wall to allow the sample to travel to the substrate;
directing a light of a particular wavelength that is suitable for Raman spectroscopy from the open end of the well through the sample of the bodily fluid to interact with the substrate;
receiving reflected light from the substrate after passing through the sample; and
analyzing the reflected light to detect a type of the virus.

16. The method of claim 15, wherein the sample is a bodily fluid.

17. The method of claim 16, wherein the virus titer in the sample is within the range of 10⁹ to 100 copies/ml saliva.

18. The method of claim 15, wherein directing light through the sample comprises:
placing the cartridge in a Raman Spectrometer.

19. The method of claim 15, wherein: directing light through the sample comprises:
aligning a handheld Raman Spectrometer over the well.

20. The method of claim 15, wherein: directing light through the sample comprises:
scanning across the well of the cartridge with x and y movement of the cartridge using a moving cartridge holder platform.

21. The method of claim 15, wherein receiving reflected light comprises:
collecting, via a detector, the reflected light to measure Raman displacement in a range from 800 cm to 3200 cm.

22. The method of claim 15, wherein: analyzing the reflected light comprises:
capturing displacements in Raman spectrum of the reflected light caused due to molecular interaction between the virus and the substrate.

23. The method of claim 22, wherein analyzing the reflected light comprises:
measuring intensity of each Raman shift or displacement within the Raman spectrum.

24. The method of claim 22, further comprising:
receiving multiple samples of the bodily fluid, wherein the cartridge comprises a plurality of wells, each well being spaced and isolated from each other, wherein each sample of the multiple samples is received within a well of the plurality of wells in the cartridge;
directing the light over each of the wells through the sample within the well; and
analyzing the reflected light received from each of the plurality of well to detect different types of viruses.

25. A system for detecting a virus, the system comprising:
a cartridge comprising:
a base;
a cover sealing coupled to the base sealing coupled to the cover forming an inner compartment therebetween, the cover comprising a well having a hydrophobic wall and a larger bore size at a top side than a bore size at a bottom side to hold a sample of bodily fluid within the well in contact with and above a substrate used to detect a virus;
a sealing element disposed within the inner compartment around the well and above the substrate; and
a Raman spectrometer configured to:
direct light of a particular wavelength toward the well;
receive reflected light from the substrate after passing through the sample of bodily fluid; and
analyze a spectrum of the reflected light to determine a type of the virus.

26. The system of claim 25, wherein the hydrophobic wall of the well comprises a first top diameter in a range from 0.5 mm to 7 mm and a second bottom diameter in a range from 0.5 mm to 5 mm to facilitate generation of Raman spectrum by a Raman spectrometer.
